(19) United States
(12) Patent Application Publication
(10) Pub. No.: US 2025/0249257 A1
(43) Pub. Date: Aug. 7, 2025

(54) METHOD AND APPARATUS FOR ECAP-BASED DYNAMIC STIMULATION PROGRAMMING

(71) Applicant: Boston Scientific Neuromodulation Corporation, Valencia, CA (US)

(72) Inventors: Jessica Block, Los Angeles, CA (US); Andrew James Haddock, Los Angeles, CA (US)

(21) Appl. No.: 19/022,006

(22) Filed: Jan. 15, 2025

Related U.S. Application Data
(60) Provisional application No. 63/549,918, filed on Feb. 5, 2024.

(51) Int. Cl. A61N 1/36

(52) U.S. Cl. CPC A61N 1/36167; A61N 1/36139

(57) ABSTRACT

A system for delivering neurostimulation to a patient using a stimulation device may include a programming control circuit and a stimulation programming circuit. The programming control circuit may be configured to generate information for programming the stimulation device to control the delivery of the neurostimulation according to a dynamic stimulation pattern defined by stimulation parameters including at least one time-varying stimulation parameter. The stimulation programming circuit includes a dynamic pattern composer, which may be configured to determine the dynamic stimulation pattern based on a targeted modulation of a neural response by the neurostimulation according to the dynamic stimulation pattern. The neural response is a response of the patient to the delivery of the neurostimulation.

### CLAIM OF PRIORITY

This application claims the benefit of U.S. Provisional Application No. 63/549,918, filed on Feb. 5, 2024, which is hereby incorporated by reference in its entirety.

### TECHNICAL FIELD

This document relates generally to neurostimulation and more particularly to a system that can deliver neurostimulation according to dynamic stimulation patterns and programming the dynamic stimulation patterns based on targeted modulation of neural responses such as evoked compound action potentials (ECAPs).

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered to a patient in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation controlled using a pattern of neurostimulation pulses defined by constant stimulation parameters has been applied to effectively treat various disorders, while use of time-varying stimulation parameters can more closely resemble natural neural activities and hence have the potential of providing for increased therapeutic effectiveness, when the stimulation parameters are properly programmed to utilize the beneficial effects of neurostimulation using one or more time-varying stimulation parameters.

### SUMMARY

An Example (e.g., “Example 1”) of a system for delivering neurostimulation to a patient using a stimulation device is provided. The system may include a programming control circuit and a stimulation programming circuit. The programming control circuit may be configured to generate information for programming the stimulation device to control the delivery of the neurostimulation according to a dynamic stimulation pattern defined by stimulation parameters including at least one time-varying stimulation parameter. The stimulation programming circuit includes a dynamic pattern composer, which may be configured to determine the dynamic stimulation pattern based on a targeted modulation of a neural response by the neurostimulation according to the dynamic stimulation pattern. The neural response is a response of the patient to the delivery of the neurostimulation.

In Example 2, the subject matter of Example 1 may optionally be configured such that the programming control circuit is further configured to generate the information for programming the stimulation device to control the delivery of the neurostimulation according to a stimulation program including multiple stimulation patterns and a schedule specifying an onset time and a duration of each stimulation pattern of the multiple stimulation patterns, the multiple stimulation patterns defined by the stimulation parameters and including the dynamic stimulation pattern, and the stimulation programming circuit is configured to determine the stimulation program.

In Example 3, the subject matter of Example 2 may optionally be configured such that the stimulation programming circuit is configured to determine the schedule for timing each stimulation pattern of the multiple stimulation patterns for an on-period followed by an off-period. The on-period is a time interval during which the neurostimulation is to be delivered according to each stimulation pattern. The off-period is another time interval during which no neurostimulation is to be delivered.

In Example 4, the subject matter of any one or any combination of Examples 1 to 3 may optionally be configured to include a stimulation device and a programming device configured to program the stimulation device and including the programming control circuit and the stimulation programming circuit. The stimulation device is configured to sense a neural signal including the neural response. The dynamic pattern composer is configured to allow for determination of one or more features of the neural response to be modulated by the neurostimulation according to the dynamic stimulation pattern.

In Example 5, the subject matter of Example 4 may optionally be configured such that the stimulation device includes an implantable stimulator, and the programming device includes an external programming device configured to be communicatively coupled to the implantable stimulator via telemetry.

In Example 6, the subject matter of Example 4 may optionally be configured such that the stimulation device includes an external test stimulator that is non-implantable.

In Example 7, the subject matter of any one or any combination of Examples 1 to 6 may optionally be configured such that the dynamic pattern composer is configured to receive input parameters defining the targeted modulation of the neural response by the neurostimulation according to the dynamic stimulation pattern and to determine the dynamic stimulation pattern based on the input parameters.

In Example 8, the subject matter of Example 7 may optionally be configured such that the dynamic pattern composer is configured to receive a neural response parameter of the input parameters by selecting the neural response parameter from a plurality of neural response parameters to be modulated by the neurostimulation according to the dynamic stimulation pattern. The neural response parameters are each a measure of the neural response and derivable from a sensed neural signal including the neural response.

In Example 9, the subject matter of Example 8 may optionally be configured such that the dynamic pattern composer is further configured to receive one or more quantitative parameters of the input parameters. The one or more quantitative parameters define a degree to which the neural response parameter is to be modulated by the neurostimulation.

In Example 10, the subject matter of Example 9 may optionally be configured such that the dynamic pattern composer is configured to receive an evoked compound action potential (ECAP) parameter as the neural response parameter. The ECAP parameter are measurable from one or more ECAP features each being a morphological feature of ECAPs in the sensed neural signal.

In Example 11, the subject matter of Example 10 may optionally be configured such that the dynamic pattern composer is configured to receive a selection of the ECAP parameter from a plurality of ECAP parameters selected from a group consisting of an amplitude between two ECAP features of the one or more ECAP features, a time interval between a stimuli of the neurostimulation and an ECAP feature of the one or more ECAP features, a time interval between two ECAP features of the one or more ECAP features, a curve length being the length of the sensed neural signal between two ECAP features of the one or more ECAP features, an area under the curve (AUC) being an area under a segment of the sensed neural signal between two ECAP features of the one or more ECAP features, and a conduction velocity measured using a feature of the one or more ECAP features.

In Example 12, the subject matter of Example 10 may optionally be configured such that the dynamic pattern composer is further configured to receive the one or more quantitative parameters including a strength of the neural response, a level of dynamic nature of the neural response, and smoothness of the neural response. The smoothness of the neural response is defined by a modulation function to be applied to produce the time-varying stimulation parameter by modulating a stimulation parameter of the stimulation parameters defining the dynamic stimulation pattern.

In Example 13, the subject matter of any one or any combination of Examples 8 to 12 may optionally be configured such that the dynamic pattern composer is configured to determine the dynamic stimulation pattern with at least one stimulation parameter of the stimulation parameters being modulated using the input parameters and a decision tree mapping plurality of neural response parameters to stimulation parameters to be modulated and types of modulation.

In Example 14, the subject matter of Example 13 may optionally be configured such that the dynamic pattern composer is further configured to receive a neural element to activate as an additional input parameter of the input parameters and to determine the dynamic stimulation pattern for activating the neural element by delivering the neurostimulation according to the dynamic stimulation pattern using the input parameters and a decision tree mapping plurality of neural response parameters to stimulation parameters to be modulated and types of modulation for the neural element.

In Example 15, the subject matter of Example 13 may optionally be configured such that the dynamic pattern composer is further configured to receive multiple neural elements to activate and an order of activation of the multiple neural elements as additional input parameters of the input parameters and to determine the dynamic stimulation pattern for activating each neural element of the multiple neural elements by delivering the neurostimulation according to the dynamic stimulation pattern using the input parameters and a decision tree mapping plurality of neural response parameters to stimulation parameters to be modulated and types of modulation for the each neural element. The dynamic stimulation pattern includes windows each associated with one neural element of the multiple neural elements to activate and temporally arranged according to the order of activation of the multiple neural elements.

An example (e.g., “Example 16”) of a method for delivering neurostimulation to a patient using a stimulation device is also provided. The method may include: determining, using a processor of a programming device, a dynamic stimulation pattern based on a targeted modulation of a neural response by the neurostimulation according to the dynamic stimulation pattern; and programming a stimulation device, using the programming device, to deliver the neurostimulation and control the delivery of the neurostimulation according to the determined dynamic stimulation pattern. The dynamic stimulation pattern is defined by stimulation parameters including at least one time-varying stimulation parameter. The neural response is a response of the patient to the delivery of the neurostimulation.

In Example 17, the subject matter of determining the dynamic stimulation pattern as found in Example 16 may optionally include: receiving input parameters defining the targeted modulation of the neural response by the neurostimulation, and determining the dynamic stimulation pattern based on the input parameters.

In Example 18, the subject matter of receiving the input parameters as found in Example 17 may optionally include receiving a neural response parameter selected from a plurality of neural response parameters to be modulated by the neurostimulation. The neural response parameters are each a measure of the neural response and derivable from a sensed neural signal including the neural response.

In Example 19, the subject matter of receiving the input parameters as found in Example 18 may optionally further include receiving one or more quantitative parameters defining a degree to which the neural response parameter is to be modulated by the neurostimulation.

In Example 20, the subject matter of receiving the neural response parameter as found in Example 19 may optionally include receiving an evoked compound action potential (ECAP) parameter. The ECAP parameter is measurable from one or more ECAP features each being a morphological feature of ECAPs in the sensed neural signal.

In Example 21, the subject matter of receiving the one or more quantitative parameters as found in Example 19 may optionally include receiving a modulation function to be applied to produce the time-varying stimulation parameter by modulating a stimulation parameter of the stimulation parameters defining the dynamic stimulation pattern.

In Example 22, the subject matter of determining the dynamic stimulation pattern as found in any one or any combination of Examples 17 to 21 may optionally include determining the dynamic stimulation pattern with at least one stimulation parameter of the stimulation parameters being modulated using the input parameters and a decision tree mapping the plurality of neural response parameters to stimulation parameters to be modulated and types of modulation.

In Example 23, the subject matter of receiving the input parameters as found in Example 22 may optionally further include receiving a neural element to activate, and the subject matter of determining the dynamic stimulation pattern as found in Example 22 may optionally include determining the dynamic stimulation pattern for activating the neural element by delivering the neurostimulation according to the dynamic stimulation pattern using the input parameters and a decision tree mapping plurality of neural response parameters to stimulation parameters to be modulated and types of modulation for the neural element.

In Example 24, the subject matter of receiving the input parameters as found in Example 22 may optionally further include receiving multiple neural elements to activate and an order of activation of the multiple neural elements, and the subject matter of determining the dynamic stimulation pattern as found in Example 22 may optionally include determining the dynamic stimulation pattern for activating each neural element of the multiple neural elements by delivering the neurostimulation according to the dynamic stimulation pattern using the input parameters and a decision tree mapping plurality of neural response parameters to stimulation parameters to be modulated and types of modulation for the each neural element. The dynamic stimulation pattern includes windows each associated with one neural element of the multiple neural elements to activate and temporally arranged according to the order of activation of the neural elements.

An example (e.g., “Example 25”) of a non-transitory computer-readable storage medium including instructions is also provided. The instructions, which when executed by a system, cause the system to perform a method for delivering neurostimulation from a stimulation device to a patient. The method may include determining a dynamic stimulation pattern based on a targeted modulation of a neural response by the neurostimulation according to the dynamic stimulation pattern, and programming the stimulation device to deliver the neurostimulation and to control the delivery of the neurostimulation according to the determined dynamic stimulation pattern. The dynamic stimulation pattern is defined by stimulation parameters including at least one time-varying stimulation parameter. The neural response is a response of the patient to the delivery of the neurostimulation.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable pulse generator (IPG) and an implantable lead system, such as an example implementation of the stimulation device and lead system of FIG. 2.

FIG. 5 illustrates an embodiment of an IPG and an implantable lead system, such as the IPG and lead system of FIG. 4, arranged to provide neurostimulation to a patient.

FIG. 6 illustrates an embodiment of portions of a neurostimulation system.

FIG. 7 illustrates an embodiment of an implantable stimulator and one or more leads of an implantable neurostimulation system, such as the implantable neurostimulation system of FIG. 6.

FIG. 8 illustrates an embodiment of an external programming device of an implantable neurostimulation system, such as the implantable neurostimulation system of FIG. 6.

FIG. 9 illustrates an embodiment of a system for programming a stimulation device, such as may be implemented in the neurostimulation system of FIG. 1.

FIGS. 10A-D illustrate examples of tonic and dynamic stimulation patterns and their effects on neural responses, with FIG. 10A showing an example with a tonic stimulation pattern, FIG. 10B showing an example with an amplitude-modulated dynamic stimulation pattern, FIG. 10C showing an example with a frequency-modulated dynamic stimulation pattern, and FIG. 10D showing a graph of a neural response parameter plotted against a stimulation frequency for the stimulation patterns of FIGS. 10A-C.

FIG. 11 illustrates an embodiment of a method for determining a dynamic stimulation pattern, such as may be performed by the system of FIG. 9.

FIG. 12 illustrates another embodiment of a method for determining a dynamic stimulation pattern, such as may be performed by the system of FIG. 9.

FIG. 13 illustrates examples of evoked compound action potential (ECAP) features of a neural signal and examples of neural response parameters produced using the ECAP features.

FIG. 14 illustrates an embodiment of a method for determining a modulation of a stimulation parameter in a dynamic stimulation pattern based on an ECAP parameter to be modulated by neurostimulation delivered according to the dynamic stimulation pattern.

FIG. 15A illustrates an example of an ECAP, and FIG. 15B illustrates an example of the ECAP of FIG. 15A being decomposed to show components of the ECAP attributed to different fiber types.

FIG. 16 illustrates an embodiment of a method for determining a dynamic stimulation pattern for activating a specified neural element, such as may be performed by the system of FIG. 9.

FIG. 17 illustrates an embodiment of a method for determining a modulation of a stimulation parameter in a dynamic stimulation pattern for a specified neural element, such as may be used in the method of FIG. 16.

FIG. 18 illustrates an embodiment of a method for determining a dynamic stimulation pattern for activating multiple specified neural elements in a specified order, such as may be performed by the system of FIG. 9.

FIG. 19 illustrates an example of a dynamic stimulation pattern determined using the method of FIG. 18.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, a neurostimulation system that can deliver neurostimulation according to dynamic stimulation patterns and program dynamic stimulation patterns based on their intended effects on sensed neural responses of a patent. In various embodiments, the neuromodulation system can include an implantable device configured to deliver neurostimulation (also referred to as neuromodulation) therapies, such as spinal cord stimulation (SCS), deep brain stimulation (DBS), peripheral nerve stimulation (PNS), and vagus nerve stimulation (VNS), and one or more external devices configured to program or adjust the implantable device for its operations and monitor the performance of the implantable device. In this document, unless noted otherwise, a “patient” includes a person receiving treatment delivered from, and/or monitored using, a neurostimulation system according to the present subject matter. A “user” includes a physician, other caregiver who examines and/or treats the patient using the neurostimulation system, or other person who participates in the examination and/or treatment of the patient using the neurostimulation system (e.g., a technically trained representative, a field clinical engineer, a clinical researcher, or a field specialist from the manufacturer of the neurostimulation system).

Delivery of neurostimulation pulses controlled using stimulation parameters programmed to constant (not time-varying) values has been effective in treating various conditions, such as in treating pain using SCS. Dynamic stimulation patterns defined by time-varying stimulation parameters can be applied to produce a wider range of responses in a patient that can be used to improve therapeutic effectiveness for the patient. Neural signals, such as neural signals can be sensed to indicate such responses in the patient, such as neural signals including evoked compound action potentials (ECAPs) sensed during SCS. However, dynamic stimulation patterns are defined by a wider range of stimulation parameters and hence substantially more complicated to program when compared to stimulation patterns defined by constant stimulation parameters. Thus, there is a need for programming dynamic stimulation patterns to utilize their clinical benefits. For example, an existing programming device may not associate effects in neural recruitment by neurostimulation with different stimulation parameters, while it is desirable to determine the stimulation parameters with knowledge of their potential effects. Such potential effects of stimulation parameters can be reflected in various ECAP parameters (e.g., strength and/or timing information measured from a sensed neural signal including ECAPs).

In this document, a “stimulation pattern” includes a pattern of neurostimulation pulses defined by stimulation parameters. A “tonic stimulation pattern” (also known as “tonic pulse sequence”, “tonic pulse train”, and the like) includes a pattern of neurostimulation pulses defined by stimulation parameters having constant values. A “dynamic stimulation pattern” (also known as “dynamic pulse sequence”, “dynamic pulse train”, and the like) includes a pattern of neurostimulation pulses defined by stimulation parameters including at least one stimulation parameter having time-varying values. One example of a dynamic stimulation pattern includes a “modulated stimulation pattern” (also known as “modulated pulse sequence”, “modulated pulse train, and the like), in which one or more of the stimulation parameters are each modulated by a modulation function. Examples of such modulated pulse sequence are discussed in U.S. patent application Ser. No. 17/530,236, “METHOD AND APPARATUS FOR GENERATING MODULATED NEUROSTIMULATION PULSE SEQUENCE”, filed on Nov. 18, 2021, published as US 2022/0184400 A1, assigned to Boston Scientific Neuromodulation Corporation, which is incorporated herein by reference in its entirety.

The present subject matter determines dynamic stimulation patterns based on their targeted effects on neural responses detectable from sensed biopotential signals (e.g., neural signals including ECAPs). A “composer” is provided for composing (i.e. creating and/or editing) neurostimulation pulses with dynamic (i.e., time-varying) stimulation parameters (e.g., pulse amplitude, pulse width, pulse rate, and the like. This neural response-based approach to dynamic stimulation pattern composition leverages sensing and biomarkers of the patients to improve efficacy of neurostimulation therapies, such as SCS. While SCS is discussed as an example of therapy and ECAP parameters are discussed as an example of sensing and biomarkers for controlling the therapy, the present subject matter is applicable to any neurostimulation therapy that can be controlled using one or more sensed signals.

Various aspects of the present subject matter include, but are not limited to, a workflow of sensing and stimulation during programming of a neurostimulation device for a patient, a user interface allowing for display of relevant information including neural signal (e.g., ECAP) recordings and neural response parameters (e.g., ECAP parameters), and controls of user interface for creating and adjusting dynamic stimulation patterns.

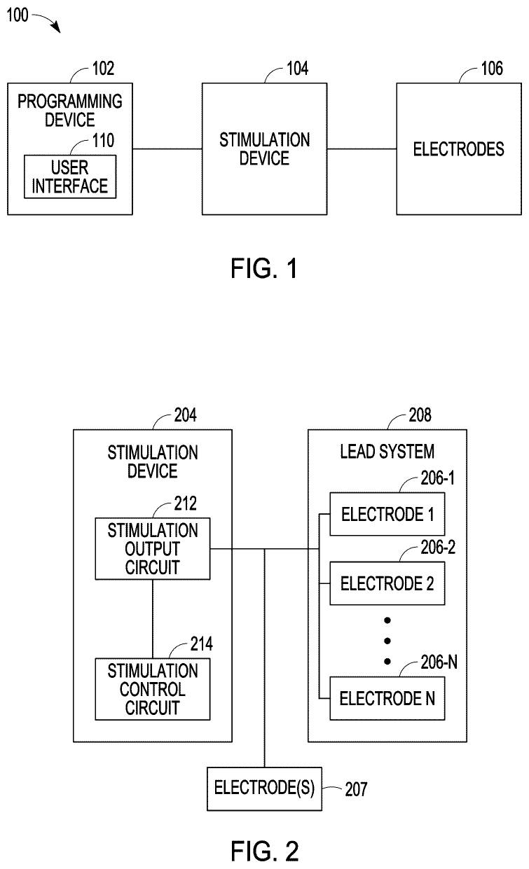

FIG. 1 illustrates an embodiment of a neurostimulation system 100. System 100 includes electrodes (also referred to as contacts) 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for spinal cord stimulation (SCS) applications. Such SCS configuration includes various features that may simplify the task of the user in programming stimulation device 104 for delivering SCS to the patient, such as the features discussed in this document.

FIG. 2 illustrates an embodiment of a stimulation device 204 and a lead system 208, such as may be implemented in neurostimulation system 100. Stimulation device 204 represents an example of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses from stimulation output circuit 212 using the plurality of stimulation parameters, which specifies a pattern of the neurostimulation pulses. Lead system 208 includes one or more leads each configured to be electrically connected to stimulation device 204 and a plurality of electrodes 206 (also referred to as an electrode array in this document) distributed in the one or more leads. The plurality of electrodes 206 includes electrode 206-1, electrode 206-2, . . . electrode 206-N, each being a single electrically conductive contact providing for an electrical interface between stimulation output circuit 212 and tissue of the patient (and therefore also referred to as a contact), where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to stimulation device 204, such as one or more electrodes each being a portion of or otherwise incorporated onto a housing of stimulation device 204. Monopolar stimulation uses a monopolar electrode configuration with one or more electrodes selected from electrodes 206 and at least one electrode from electrode(s) 207. Bipolar stimulation uses a bipolar electrode configuration with two electrodes selected from electrodes 206 and none of electrode(s) 207. Multipolar stimulation uses a multipolar electrode configuration with multiple (two or more) electrodes selected from electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

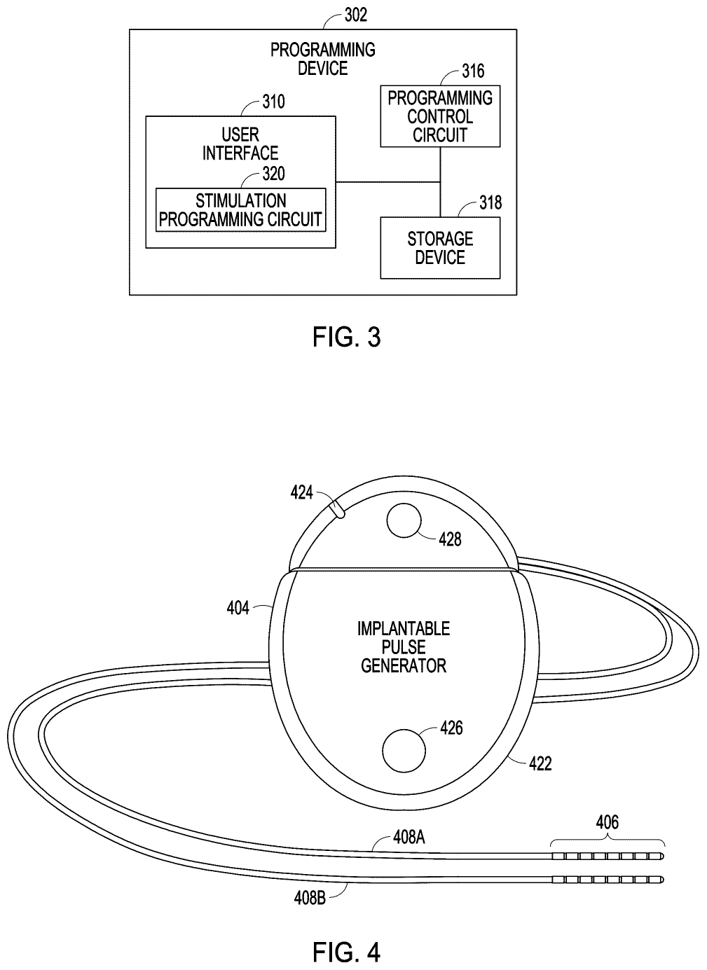

FIG. 3 illustrates an embodiment of a programming device 302, such as may be implemented in neurostimulation system 100. Programming device 302 represents an example of programming device 102 and includes a storage device 318, a programming control circuit 316, and a user interface 310. Programming control circuit 316 generates the plurality of stimulation parameters that controls the delivery of the neurostimulation pulses according to a specified neurostimulation program that can define, for example, stimulation waveform and electrode configuration. User interface 310 represents an example of user interface 110 and includes a stimulation programming circuit 320. Storage device 318 stores information used by programming control circuit 316 and stimulation programming circuit 320, such as information about a stimulation device that relates the neurostimulation program to the plurality of stimulation parameters. In various embodiments, stimulation programming circuit 320 can be configured to support one or more functions allowing for programming of stimulation devices, such as stimulation device 104 including its various embodiments as discussed in this document, to control delivery of neurostimulation according to the present subject matter (e.g., using one or more dynamic stimulation patterns determined based on targeted effects on neural responses sensed from the patient).

In various embodiments, user interface 310 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “neurostimulation program” can include the pattern of neurostimulation pulses including the one or more stimulation fields, or at least various aspects or parameters of the pattern of neurostimulation pulses including the one or more stimulation fields. In various embodiments, user interface 310 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

In various embodiments, circuits of neurostimulation system 100, including its various embodiments discussed in this document, may be implemented using a combination of hardware and software. For example, the circuit of user interface 110, stimulation control circuit 214, programming control circuit 316, and stimulation programming circuit 320, including their various embodiments discussed in this document, can be implemented using an application-specific circuit constructed to perform one or more particular functions and/or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 4 illustrates an embodiment of an implantable pulse generator (IPG) 404 and an implantable lead system 408. IPG 404 represents an example implementation of stimulation device 204. Lead system 408 represents an example implementation of lead system 208. As illustrated in FIG. 4, IPG 404 that can be coupled to implantable leads 408A and 408B at a proximal end of each lead. The distal end of each lead includes electrodes 406 for contacting a tissue site targeted for electrical neurostimulation. As illustrated in FIG. 4, leads 408A and 408B each include 8 electrodes 406 at the distal end. The number and arrangement of leads 408A and 408B and electrodes 406 as shown in FIG. 4 are only an example, and other numbers and arrangements are possible. In various embodiments, the electrodes are ring electrodes. In various embodiments applying DBS or SCS, the implantable leads and electrodes may be configured by shape and size to provide electrical neurostimulation energy to a neuronal target included in the patient's brain or configured to provide electrical neurostimulation energy to target nerve cells in the patient's spinal cord.

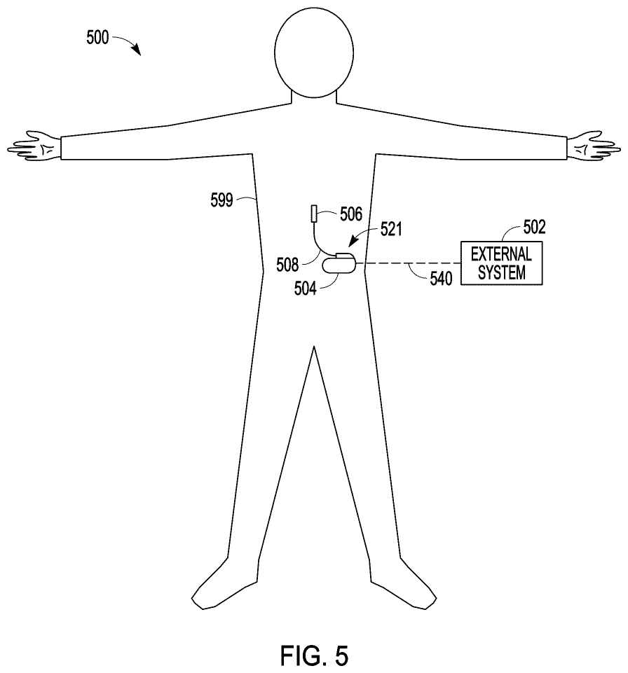

FIG. 5 illustrates an implantable neurostimulation system 500 and portions of an environment in which system 500 may be used. System 500 includes an implantable system 521, an external system 502, and a telemetry link 540 providing for wireless communication between implantable system 521 and external system 502. Implantable system 521 is illustrated in FIG. 5 as being implanted in the patient's body 599.

Implantable system 521 includes an implantable stimulator (also referred to as an implantable pulse generator, or IPG) 504, a lead system 508, and electrodes 506, which represent an example of stimulation device 204, lead system 208, and electrodes 206, respectively. External system 502 represents an example of programming device 302. In various embodiments, external system 502 includes one or more external (non-implantable) devices each allowing the user and/or the patient to communicate with implantable system 521. In some embodiments, external 502 includes a programming device intended for the user to initialize and adjust settings for implantable stimulator 504 and a remote control device intended for use by the patient. For example, the remote control device may allow the patient to turn implantable stimulator 504 on and off and/or adjust certain patient-programmable parameters of the plurality of stimulation parameters.

The sizes and shapes of the elements of implantable system 521 and their location in body 599 are illustrated by way of example and not by way of restriction. An implantable system is discussed as a specific application of the programming according to various embodiments of the present subject matter. In various embodiments, the present subject matter may be applied in programming any type of stimulation device that uses electrical pulses as stimuli, regarding less of stimulation targets in the patient's body and whether the stimulation device is implantable.

Returning to FIG. 4, the IPG 404 can include a hermetically-sealed IPG case 422 to house the electronic circuitry of IPG 404. IPG 404 can include an electrode 426 formed on IPG case 422. IPG 404 can include an IPG header 424 for coupling the proximal ends of leads 408A and 408B. IPG header 424 may optionally also include an electrode 428. Electrodes 426 and/or 428 represent embodiments of electrode(s) 207 and may each be referred to as a reference electrode. Neurostimulation energy can be delivered in a monopolar (also referred to as unipolar) mode using electrode 426 or electrode 428 and one or more electrodes selected from electrodes 406. Neurostimulation energy can be delivered in a bipolar mode using a pair of electrodes of the same lead (lead 408A or lead 408B). Neurostimulation energy can be delivered in an extended bipolar mode using one or more electrodes of a lead (e.g., one or more electrodes of lead 408A) and one or more electrodes of a different lead (e.g., one or more electrodes of lead 408B).

The electronic circuitry of IPG 404 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

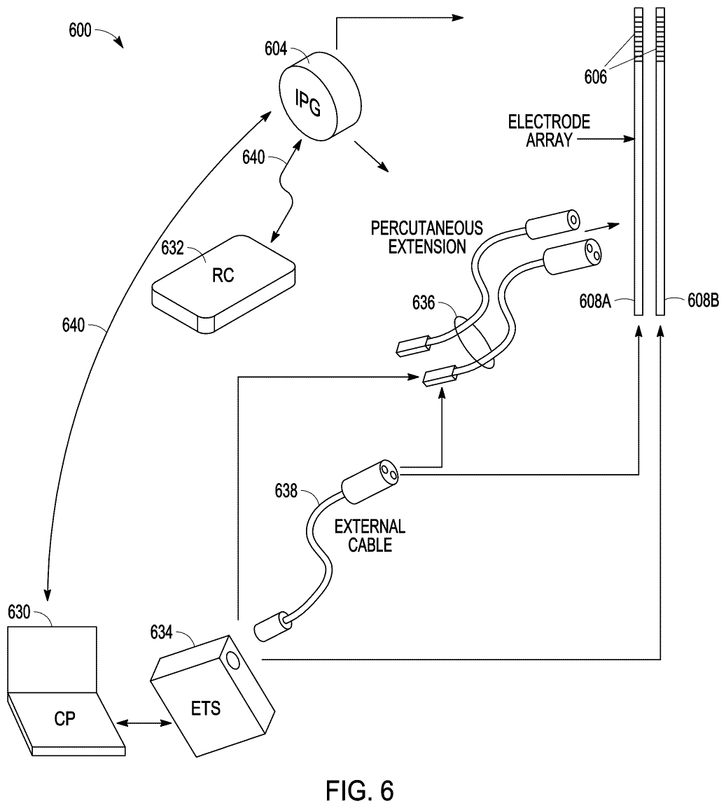

FIG. 6 illustrates an embodiment of portions of a neurostimulation system 600. System 600 includes an IPG 604, implantable neurostimulation leads 608A and 608B, an external remote controller (RC) 632, a clinician's programmer (CP) 630, and an external trial stimulator (ETS, also referred to as external trial modulator, ETM) 634. IPG 604 may be electrically coupled to leads 608A and 608B directly or through percutaneous extension leads 636. ETS 634 may be electrically connectable to leads 608A and 608B via one or both of percutaneous extension leads 636 and/or external cable 638. System 600 represents an example of system 100, with IPG 604 representing an embodiment of stimulation device 104, electrodes 606 of leads 608A and 608B representing electrodes 106, and CP 630, RC 632, and ETS 634 collectively representing programming device 102.

ETS 634 may be standalone or incorporated into CP 630. ETS 634 may have similar pulse generation circuitry as IPG 604 to deliver neurostimulation energy according to specified modulation parameters as discussed above. ETS 634 is an external device that is typically used as a preliminary stimulator after leads 408A and 408B have been implanted and used prior to stimulation with IPG 604 to test the patient's responsiveness to the stimulation that is to be provided by IPG 604. Because ETS 634 is external it may be more easily configurable than IPG 604.

CP 630 can configure the neurostimulation provided by ETS 634. If ETS 634 is not integrated into CP 630, CP 630 may communicate with ETS 634 using a wired connection (e.g., over a USB link) or by wireless telemetry using a wireless communications link 640. CP 630 also communicates with IPG 604 using a wireless communications link 640.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. IPG 604 can include the first coil and a communication circuit. CP 630 can include or otherwise electrically connected to the second coil such as in the form of a wand that can be place near IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least six feet but can be as long as allowed by the particular communication technology. RF antennas can be included, for example, in the header of IPG 604 and in the housing of CP 630, eliminating the need for a wand or other means of inductive coupling. An example is such an RF telemetry link is a Bluetooth® wireless link.

CP 630 can be used to set modulation parameters for the neurostimulation after IPG 604 has been implanted. This allows the neurostimulation to be tuned if the requirements for the neurostimulation change after implantation. CP 630 can also upload information from IPG 604.

RC 632 also communicates with IPG 604 using a wireless link 640. RC 632 may be a communication device used by the user or given to the patient. RC 632 may have reduced programming capability compared to CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by CP 630. CP 630 may communicate with the RC 632 using a wired or wireless communications link. In some embodiments, CP 630 can program RC 632 when remotely located from RC 632. In various embodiments, RC 632 can be a dedicated device or a general-purpose device configured to perform the functions of RC 632, such as a smartphone, a tablet computer, or other smart/mobile device.

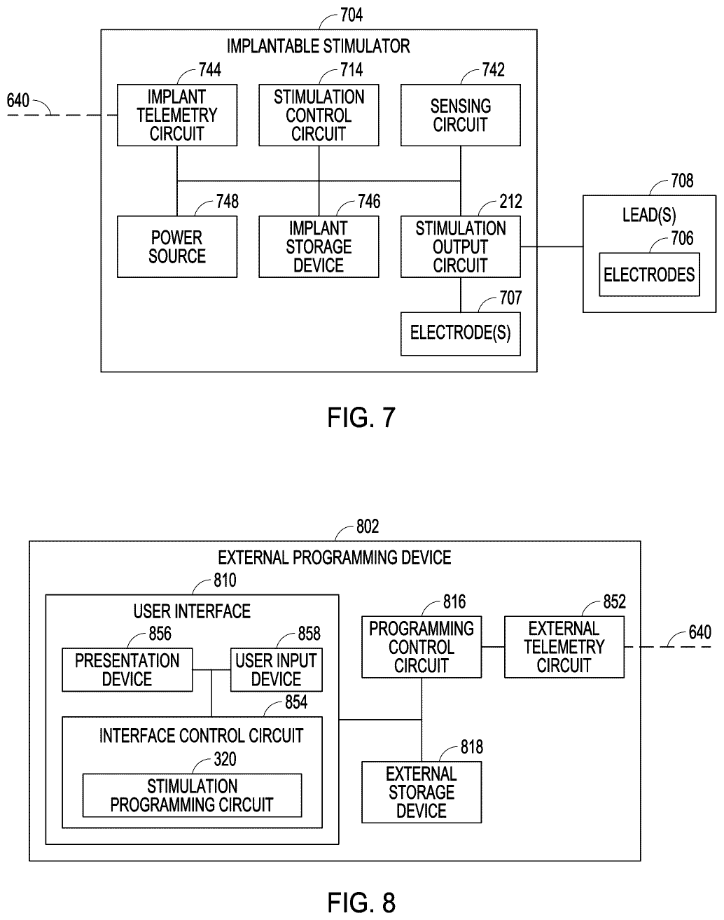

FIG. 7 illustrates an embodiment of implantable stimulator 704 and one or more leads 708 of an implantable neurostimulation system, such as implantable system 600. Implantable stimulator 704 represents an example of stimulation device 104 or 204 and may be implemented, for example, as IPG 604. Lead(s) 708 represents an example of lead system 208 and may be implemented, for example, as implantable leads 608A and 608B. Lead(s) 708 includes electrodes 706, which represents an example of electrodes 106 or 206 and may be implemented as electrodes 606.

Implantable stimulator 704 may include a sensing circuit 742 that provides the stimulator with a sensing capability, stimulation output circuit 212, a stimulation control circuit 714, an implant storage device 746, an implant telemetry circuit 744, a power source 748, and one or more electrodes 707. Sensing circuit 742 can one or more physiological signals for purposes of patient monitoring and/or feedback control of the neurostimulation. In various embodiments, sensing circuit 742 can sense one or more electrospinogram (ESG) signals using electrodes placed over or under the dura of the spinal cord, such as electrodes 706 (which can include epidural and/or intradural electrodes). In addition to one or more ESG signals, examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 706 through one or more leads 708 as well as electrodes 707 and delivers each of the neurostimulation pulses through a set of electrodes selected from electrodes 706 and electrode(s) 707. Stimulation control circuit 714 represents an example of stimulation control circuit 214 and controls the delivery of the neurostimulation pulses using the plurality of stimulation parameters specifying the pattern of neurostimulation pulses. In one embodiment, stimulation control circuit 714 controls the delivery of the neurostimulation pulses using the one or more sensed physiological signals. Implant telemetry circuit 744 provides implantable stimulator 704 with wireless communication with another device such as CP 630 and RC 632, including receiving values of the plurality of stimulation parameters from the other device. Implant storage device 746 can store one or more neurostimulation programs and values of the plurality of stimulation parameters for each of the one or more neurostimulation programs. Power source 748 provides implantable stimulator 704 with energy for its operation. In one embodiment, power source 748 includes a battery. In one embodiment, power source 748 includes a rechargeable battery and a battery charging circuit for charging the rechargeable battery. Implant telemetry circuit 744 may also function as a power receiver that receives power transmitted from an external device through an inductive couple. Electrode(s) 707 allow for delivery of the neurostimulation pulses in the monopolar mode. Examples of electrode(s) 707 include electrode 426 and electrode 418 in IPG 404 as illustrated in FIG. 4.

In one embodiment, implantable stimulator 704 is used as a master database. A patient implanted with implantable stimulator 704 (such as may be implemented as IPG 604) may therefore carry patient information needed for his or her medical care when such information is otherwise unavailable. Implant storage device 746 is configured to store such patient information. For example, the patient may be given a new RC 632 (e.g., by installing a new application in a smart device such as a smartphone) and/or travel to a new clinic where a new CP 630 is used to communicate with the device implanted in him or her. The new RC 632 and/or CP 630 can communicate with implantable stimulator 704 to retrieve the patient information stored in implant storage device 746 through implant telemetry circuit 744 and wireless communication link 640 and allow for any necessary adjustment of the operation of implantable stimulator 704 based on the retrieved patient information. In various embodiments, the patient information to be stored in implant storage device 746 may include, for example, positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in implant storage device 746 may include data transmitted to implantable stimulator 704 for storage as part of the patient information and data acquired by implantable stimulator 704, such as by using sensing circuit 742.

In various embodiments, sensing circuit 742 (if included), stimulation output circuit 212, stimulation control circuit 714, implant telemetry circuit 744, implant storage device 746, and power source 748 are encapsulated in a hermetically sealed implantable housing or case, and electrode(s) 707 are formed or otherwise incorporated onto the case. In various embodiments, lead(s) 708 are implanted such that electrodes 706 are placed on and/or around one or more targets to which the neurostimulation pulses are to be delivered, while implantable stimulator 704 is subcutaneously implanted and connected to lead(s) 708 at the time of implantation.

FIG. 8 illustrates an embodiment of an external programming device 802 of an implantable neurostimulation system, such as system 600. External programming device 802 represents an example of programming device 102 or 302, and may be implemented, for example, as CP 630 and/or RC 632. External programming device 802 includes an external telemetry circuit 852, an external storage device 818, a programming control circuit 816, and a user interface 810.

External telemetry circuit 852 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one embodiment, external telemetry circuit 852 also transmits power to implantable stimulator 704 through an inductive couple.

In various embodiments, wireless communication link 640 can include an inductive telemetry link (near-field telemetry link) and/or a far-field telemetry link (RF telemetry link). This can allow for patient mobility during programming and assessment when needed. For example, wireless communication link 640 can include at least a far-field telemetry link that allows for communications between external programming device 802 and implantable stimulator 704 over a relative long distance, such as up to about 20 meters. External telemetry circuit 852 and implant telemetry circuit 744 each include an antenna and RF circuitry configured to support such wireless telemetry.

External storage device 818 stores one or more stimulation waveforms for delivery during a neurostimulation therapy session, such as a DBS or SCS therapy session, as well as various parameters and building blocks for defining one or more waveforms. The one or more stimulation waveforms may each be associated with one or more stimulation fields and represent a pattern of neurostimulation pulses to be delivered to the one or more stimulation field during the neurostimulation therapy session. In various embodiments, each of the one or more stimulation waveforms can be selected for modification by the user and/or for use in programming a stimulation device such as implantable stimulator 704 to deliver a therapy. In various embodiments, each waveform in the one or more stimulation waveforms is definable on a pulse-by-pulse basis, and external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through which a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2 electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g., mA) allows precise dosing of current through each specific electrode. It is suited for changing the current one contact at a time to shape the stimulation like a piece of clay (pushing/pulling one spot at a time).

Programming control circuit 816 represents an example of programming control circuit 316 and generates the plurality of stimulation parameters, which is to be transmitted to implantable stimulator 704, based on a specified neurostimulation program (e.g., the pattern of neurostimulation pulses as represented by one or more stimulation waveforms and one or more stimulation fields, or at least certain aspects of the pattern). The neurostimulation program may be created and/or adjusted by the user using user interface 810 and stored in external storage device 818. In various embodiments, programming control circuit 816 can check values of the plurality of stimulation parameters against safety rules to limit these values within constraints of the safety rules. In one embodiment, the safety rules are heuristic rules.

User interface 810 represents an example of user interface 310 and allows the user to define the pattern of neurostimulation pulses and perform various other monitoring and programming tasks. User interface 810 includes a display screen 856, a user input device 858, and an interface control circuit 854. Display screen 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 810 includes a GUI. The GUI may also allow the user to perform any functions discussed in this document where graphical presentation and/or editing are suitable as may be appreciated by those skilled in the art.

Interface control circuit 854 controls the operation of user interface 810 including responding to various inputs received by user input device 858 and defining the one or more stimulation waveforms. Interface control circuit 854 includes stimulation programming circuit 320.

In various embodiments, external programming device 802 can have operation modes including a composition mode and a real-time programming mode. Under the composition mode (also known as the pulse pattern composition mode), user interface 810 is activated, while programming control circuit 816 is inactivated. Programming control circuit 816 does not dynamically updates values of the plurality of stimulation parameters in response to any change in the one or more stimulation waveforms. Under the real-time programming mode, both user interface 810 and programming control circuit 816 are activated. Programming control circuit 816 dynamically updates values of the plurality of stimulation parameters in response to changes in the set of one or more stimulation waveforms and transmits the plurality of stimulation parameters with the updated values to implantable stimulator 704.

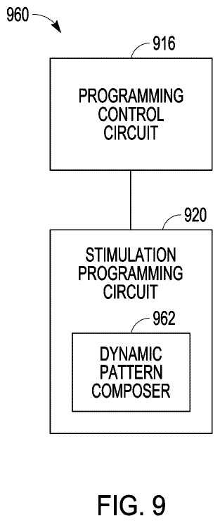

FIG. 9 illustrates an embodiment of a system 960 for programming a stimulation device, such as a stimulation device discussed above (e.g., stimulation device 104, stimulation device 204, IPG 404, IPG 504, IPG 604, ETS 634, or implantable stimulation 704). System 960 can include a programming control circuit 916 and a stimulation programming circuit 920. System 960 can be implemented in a neurostimulation system such as systems 100, 500, or 600. In various embodiments, system 960 can be implemented in an external system including one or more programming devices, such as programming device 102, programing device 302, external system 502, CP 630 and/or RC 632, or external programming device 802. For example, when system 960 is implemented in external programming device 802, programming control circuit 916 is implemented in programming control circuit 816, and stimulation programming circuit 920 is implemented in stimulation programming circuit 320. In various embodiments, system 960 can be implemented in a single device or in two or more devices.

Programming control circuit 916 can generate information for programming the stimulation device to control the delivery of the neurostimulation according to a dynamic stimulation pattern defined by stimulation parameters including at least one time-varying stimulation parameter. Stimulation programming circuit 920 includes a dynamic pattern composer 962. Dynamic pattern composer 962 can determine the dynamic stimulation pattern based on a targeted modulation of a neural response by the neurostimulation according to the dynamic stimulation pattern. The neural response is a response of the patient to the delivery of the neurostimulation.

In various embodiments, system 960 can be used to program a stimulation device that can deliver the neurostimulation to the patient and sense a neural signal including neural responses from the patient. The neural responses result from the delivered neurostimulation. For example, the sensed neural signal can include evoked compound action potentials (ECAPs) each evoked by a pulse of the neurostimulation. The targeted modulation of the neural response can be a modulation of the neural response as detected from the sensed neural signal, determined based on a desirable change of a physiological phenomenon that is reflected in the neural responses. The stimulation device can be programmed to deliver the neurostimulation according to the determined dynamic stimulation pattern to verify the target modulation including its effect on the patient. In various embodiments, system 960 can be used to program the stimulation device for multiple targeted modulations of neural responses of the patient to effect changes in various physiological phenomena as therapeutic benefits. An example of the stimulation device with sensing capability is implantable stimulator 704, which include sensing circuit 742 that can sense a neural signal (e.g., an ESG signal) that includes ECAPs using electrodes 706. The stimulation device can also be an external (non-implantable) stimulator such as ETS 634. An example of an external stimulation with sensing capabilities is discussed in U.S. Pat. No. 11,648,399, “SENSING REFERENCE ELECTRODE FOR PERCUTANEOUS NEUROMODULATION TRIALS”, assigned to Boston Scientific Neuromodulation Corporation, which is incorporated herein by reference in its entirety.

In various embodiments, programming control circuit 916 can generate information for programming the stimulation device to control the delivery of the neurostimulation according to a stimulation program. The stimulation program can specify stimulation parameters including stimulation waveform parameters defining at least one stimulation waveform of the neurostimulation and stimulation field parameters defining at least one stimulation field to which the neurostimulation is to be delivered. The stimulation program can include one or more stimulation patterns. The one or more stimulation patterns can include one or more dynamic stimulation patterns each defined by the stimulation parameters including at least one time-varying stimulation waveform parameter. The stimulation program can include multiple stimulation patterns and a schedule specifying onset and duration of each stimulation pattern of the multiple stimulation patterns. Each stimulation pattern of the multiple stimulation patterns can be applied for an on-period followed by an off-period. The on-period is a time interval during which the neurostimulation is to be delivered according to a stimulation pattern. The off-period is another time interval during which no neurostimulation is to be delivered. Stimulation programming circuit 920 can determine the stimulation program, and dynamic pattern composer 962 can determine each dynamic stimulation pattern of the stimulation program based on the neural response targeted to be modulated by the neurostimulation. To determine (or “compose”) the dynamic stimulation pattern can include creating a new dynamic stimulation pattern or adjusting a template dynamic stimulation pattern.

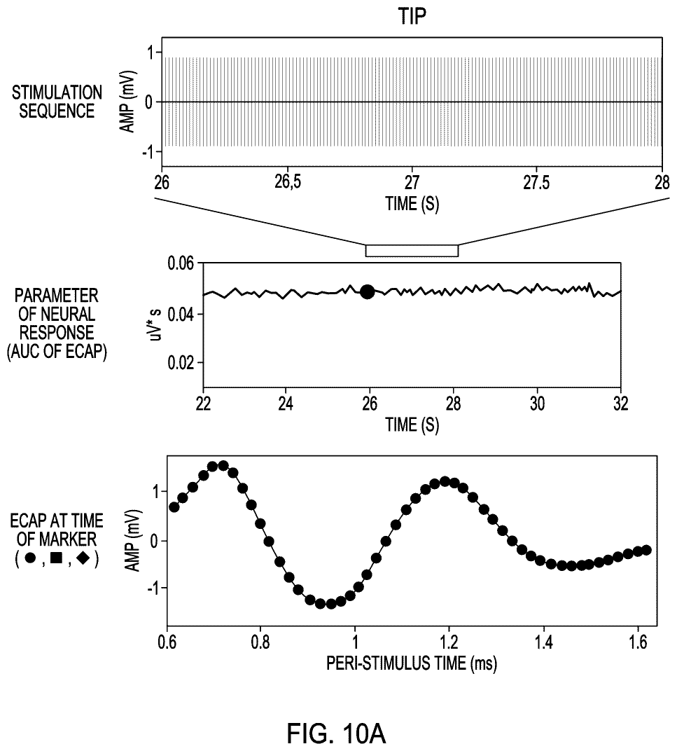

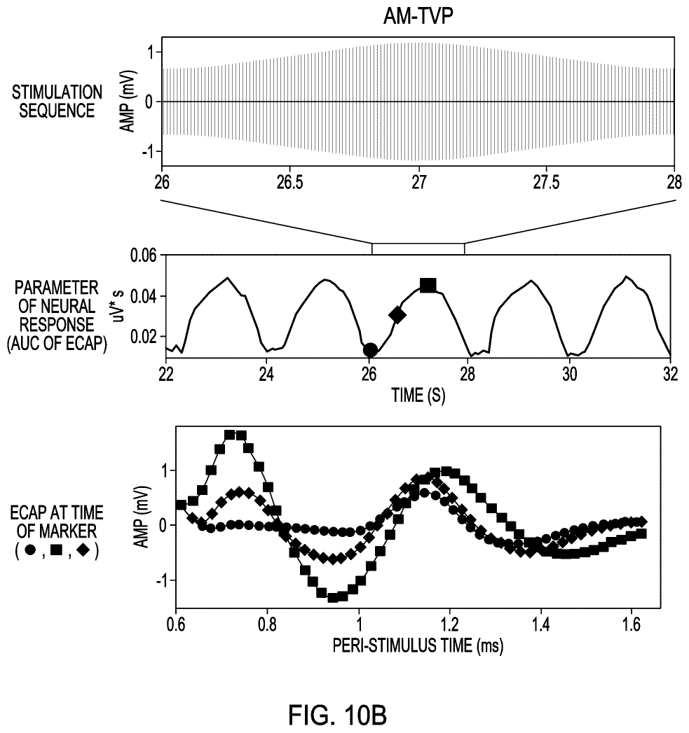

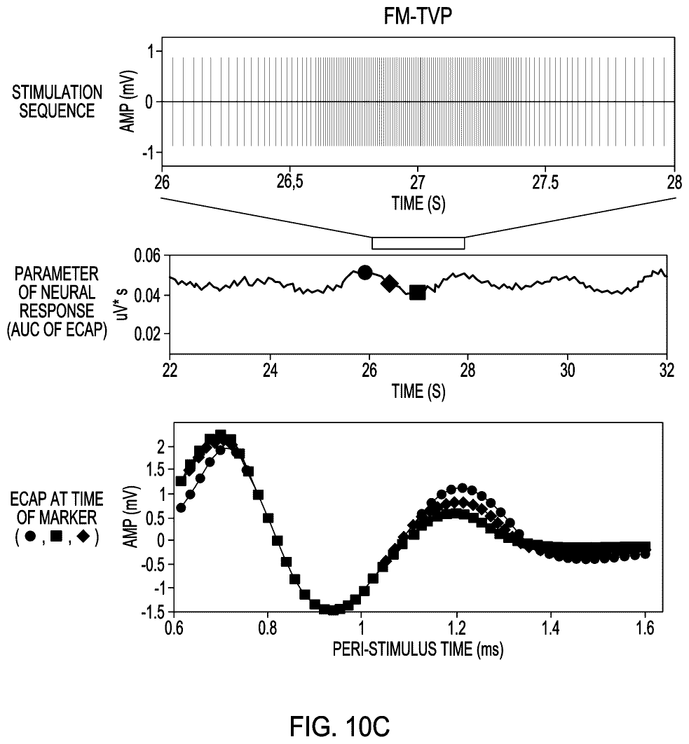

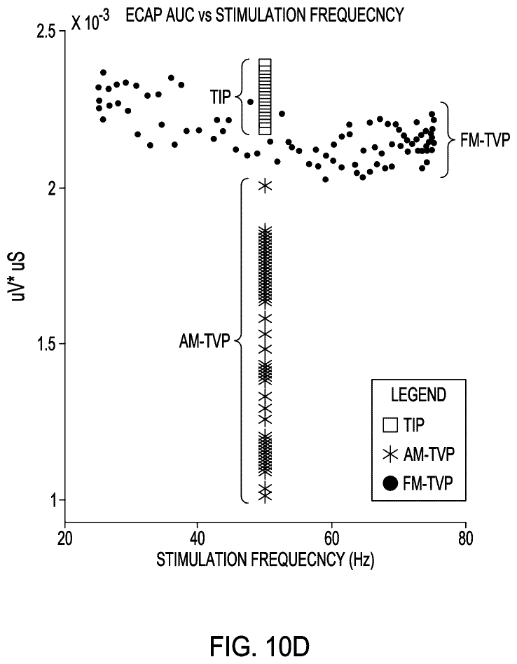

FIGS. 10A-D illustrate examples of tonic and dynamic stimulation patterns and their effects on neural responses. FIGS. 10A-C show, respectively, neurostimulation pulse sequences including TIP (time-independent pulses), AM-TVP (amplitude-modulated time-varying pulses), and FM-TVP (frequency-modulated time-varying pulses), their respective neural responses as represented by an ECAP parameter, the area under curve (AUC), and the ECAP as measured at different points of the neural responses. The TIP is an example of the tonic stimulation pattern, the AM-TVP is an example of the dynamic stimulation patterns being the tonic stimulation pattern with its pulse amplitude being modulated, and the FM-TVP is another example of the dynamic stimulation patterns being the tonic stimulation pattern with its pulse frequency (also referred to as pulse rate) being modulated. FIG. 10D shows a graph of the AUC plotted against the stimulation frequency (i.e., the pulse frequency) for the TIP, AM-TVP, and FM-TVP pulse sequences. It can be seen from FIGS. 10A-D that the dynamic stimulation patterns change the effect of neurostimulation according to the tonic stimulation pattern on the sensed ECAP features and profile differently, depending on the type of modulation of the stimulation parameter applied.

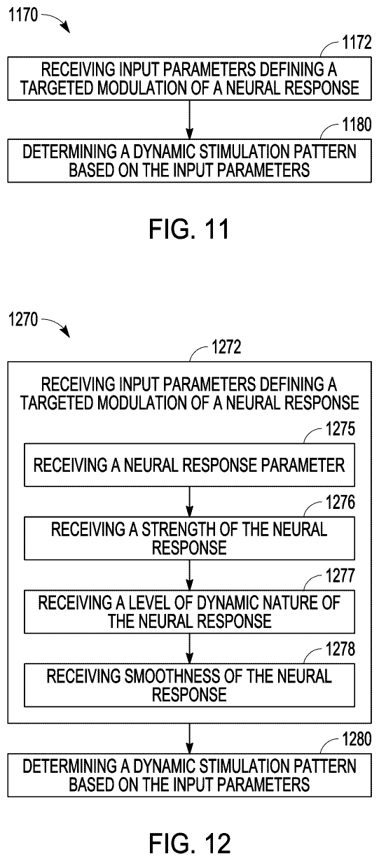

FIG. 11 illustrates an embodiment of a method 1170 for determining a dynamic stimulation pattern, such as may be performed by system 960. Dynamic pattern composer 962 can be configured for performing method 1170. In one embodiment, in which system 960 is implemented in external programming device 802, external storage device 818 is configured to be a non-transitory computer-readable storage medium including instructions, which when executed by a system (e.g., system 960 as implemented in external programming device 802), cause the system to perform method 1170.

At 1172, input parameters defining a targeted modulation of a neural response are received. The neural response is a response of the patient to delivery of neurostimulation according to a dynamic stimulation pattern. In various embodiments, the input parameters can be received from a user using a user input device (e.g., user input device 858), predetermined and stored (e.g., in external storage device 818), and/or automatically determined (e.g., by dynamic pattern composer 962 based on available information related to the patient). For example, all of the input parameters can be received from the user using the user input device, or at least some of the input parameters can be provided by a system used to (e.g., system 960) perform method 1170.

At 1180, the dynamic stimulation pattern is determined based on the input parameters. The dynamic stimulation pattern is defined by stimulation parameters including at least one time-varying stimulation parameter.

A stimulation device can be programmed to deliver the neurostimulation and to control the delivery of the neurostimulation according to the determined dynamic stimulation pattern. Examples of the stimulation device include the stimulation devices discussed above (e.g., stimulation device 104, stimulation device 204, IPG 404, IPG 504, IPG 604, ETS 634, and implantable stimulation 704).

FIG. 12 illustrates an embodiment of a method 1270 for determining a dynamic stimulation pattern, such as may be performed by system 960. Method 1270 can represent an example of method 1170 including examples of the input parameters and examples of the time-varying stimulation parameter in the dynamic stimulation pattern. Dynamic pattern composer 962 can be configured for performing method 1270. In one embodiment, in which system 960 is implemented in external programming device 802, external storage device 818 is configured to be a non-transitory computer-readable storage medium including instructions, which when executed by a system (e.g., system 960 as implemented in external programming device 802), cause the system to perform method 1270.

At 1272, input parameters defining a targeted modulation of a neural response are received. The neural response is a response of the patient to delivery of neurostimulation according to a dynamic stimulation pattern and can be detected from a neural signal sensed from the patient. In various embodiments, the input parameters can define a feature of the neural response (e.g., a neural response parameter) to be modulated and a degree to which the feature of the neural response is to be modulated. At 1275, a neural response parameter is received. The neural response parameter can be derived from the neural response. At 1276, a strength of the neural response is received. The strength can be represented by, for example, an average value of the neural response parameter. At 1277, a level of dynamic nature of the neural response is received. The level can be represented by a value range of the neural response parameter. In one example, the neural response parameter is an ECAP parameter (a measure of an ECAP resulting from a neurostimulation pulse, e.g., a peak-to-peak amplitude of the ECAP) selected from a plurality of ECAP parameters, the strength of the neural response is an average value of the ECAP parameter (e.g., 30 microvolts), and the level of dynamic nature of the neural response is a value range of the ECAP parameter (e.g., 10-50 microvolts). At 1278, smoothness of the neural response is received. The smoothness can be represented by a modulation function applied to a stimulation parameter of the dynamic stimulation pattern. The modulation function can be selected from a plurality of modulation functions (e.g., as supported by dynamic stimulation composer 962) for modulating the stimulation parameter. Examples of the modulation function include random, pseudo-random, sinusoidal, triangular, Poisson distribution, Gaussian distribution, and any other standard or custom functions.

At 1280, the dynamic stimulation pattern is determined based on the input parameters. The dynamic stimulation pattern is defined by stimulation parameters including at least one time-varying stimulation parameter. In various embodiments, the dynamic stimulation pattern is determined based on the input parameters and a decision tree mapping the input parameters to stimulation parameters to be modulated and types of the modulation. In the example in which the neural response parameter is the peak-to-peak amplitude of the ECAP, the dynamic stimulation patterns determined based on received at 1275, 1276, 1277, and 1278 includes a neurostimulation pulse sequence with a pulse amplitude to be set and modulated by the sinusoidal modulation function to result in the peak-to-peak amplitude of ECAPs ranging from 10-50 microvolts, with the averaged value of 30 microvolts. The pulse amplitude is selected to be modulated according to a decision tree. The neural response parameter and the decision tree are further discussed below.

FIG. 12 illustrates examples of ECAP features of a neural signal and examples of neural response parameters produced using the ECAP features. The neural response parameter can each be used to indicate a physiological phenomenon that can be targeted to be modulated by the neurostimulation. For example, morphological features of ECAP indicate types of nerve fibers activated, effects of electrodes relative to the spinal cord, etc. The reason for selecting a particular neural response parameter (an ECAP parameter in the example of FIG. 12) is the intention for changing the physiological phenomenon indicated by that parameter for therapeutic benefits.

The signal shown in FIG. 12 is an example of a neural response that is evoked by a neurostimulation pulse and includes an ECAP indicative of dorsal column response (e.g., as seen on an electrospinographic signal). Morphological features of the ECAP (herein referred to as “ECAP features”) that can be used in the present subject matter include, but are not limited to:
N1: the first negative peak in an evoked response that is correlated to the response of faster fibers such as A-beta fibers and myelinated fibers; and
P2: the second positive peak in the evoked response that is correlated with response of slower fibers such as A-delta fibers or C fibers.

Various neural response parameters can be derived from characteristics of the evoked responses as seen on the neural signal. In various embodiments, one or more neural response parameters can be measured from the neural signal using the ECAP features. Examples of the one or more neural response parameters generated by detecting and measuring ECAP features for analyzing neural activation and/or controlling delivery of neurostimulation include:
N1 amplitude: amplitude of N1 (measured from a base line, e.g., 0 V);
P1 amplitude: amplitude of P1 (measured from the base line);
N1-P2 amplitude: the difference between amplitudes of N1 and P2 (also referred to as the peak-to-peak amplitude);
N1-P2 latency: time interval between N1 and P2;
N1 latency: time interval between start of recording frame (e.g., the neurostimulation pulse) and N1;
P2 latency: time interval between start of recording frame (e.g., the neurostimulation pulse) and P2;
Curve length (CL): curve length measured from the sensed neural signal between N1 and the end of the ECAP (when the neural signal returns to its baseline);
Area under the curve (AUC): the area between the sensed neural signal and a baseline, measured between N1 and P2;
Inter-ECAP interval: time interval between N1 of an ECAP and N1 of the immediately preceding ECAP; and
Conduction velocity.

The conduction velocity can be measured from ECAP by (1) calculating from a known distance between two electrodes placed over the spinal cord and the times for an ECAP feature to travel between the two electrodes as can be sensed using the two electrodes, or (2) calculating from a known distance between a stimulation electrode and a sensing electrode and time when an ECAP is sensed using the sensing electrode relative to the time when the pulse evoking the ECAP is delivered to the stimulating electrode. It is noted that method (1) may be preferred over method (2), which is more likely affected by various physiological factors, under some circumstances.

In addition to or in place of the time-domain parameters above, the one or more neural response parameters can also include, for example:
Spectral power parameters (e.g. peak amplitude of each ECAP feature and/or total AUC of ECAP power band from 500 Hz to 3000 Hz);
Parseval power parameters (integral of the spectrum squared over a relevant power band); and
Other metrics of energy or power.

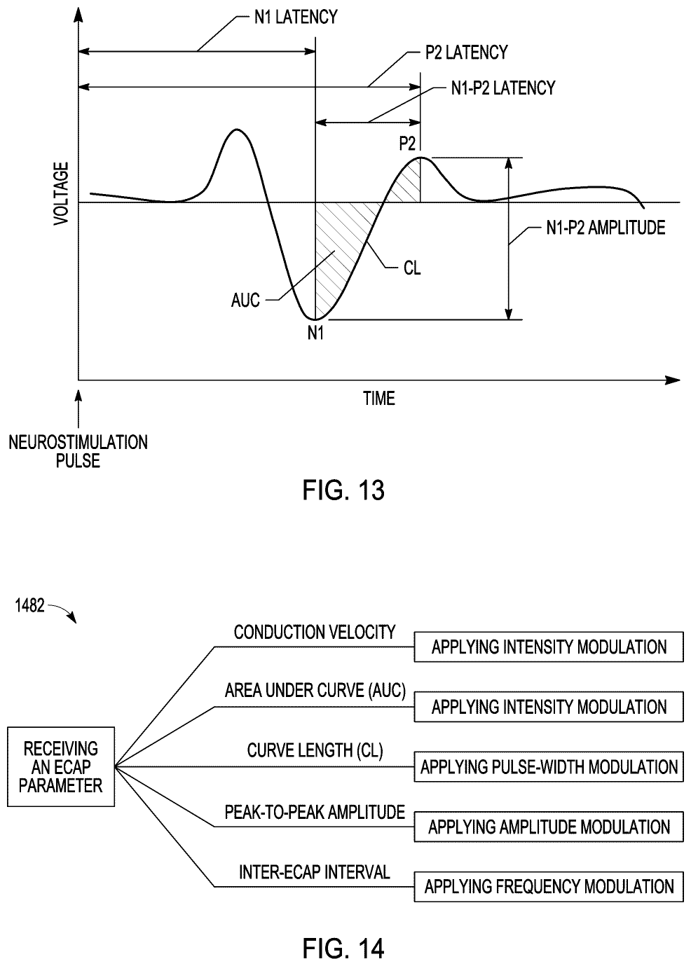

In various embodiments, one or more of these neural response parameters can be selected to determine a dynamic stimulation pattern based on one or more physiological phenomena intended to be modulated by the neurostimulation delivered according to the dynamic stimulation pattern. While the ECAP features and neural response parameters illustrated in FIG. 13 are discussed as examples, other ECAP features and neural response parameters, as well as other types of neural responses and their measures can be suitable and used in the present subject matter as determined by those skilled in the art.

FIG. 14 illustrates an embodiment of a method 1482 for determining a modulation of a stimulation parameter in a dynamic stimulation pattern based on an ECAP parameter to be modulated by neurostimulation delivered according to the dynamic stimulation pattern. Method 1482 can represent an example of the decision tree used in step 1280 of method 1270. To modulate the neural response parameter as intended, decision tree 1482 can be used with the input parameters to determine the dynamic stimulation pattern, which is defined by the stimulation parameters including at least one modulated stimulation parameter.

According to method (decision tree) 1482, an ECAP parameter is received as the neural response parameter to be modulated by neurostimulation. If the ECAP parameter is the conduction velocity, an intensity of the neurostimulation (e.g., a pulse amplitude, a pulse width, and/or a pulse frequency) is to be modulated. If the ECAP parameter is the AUC (which is highly correlated to the peak-to-peak amplitude), an intensity of the neurostimulation (e.g., a pulse amplitude, a pulse width, and/or a pulse frequency) is to be modulated. If the ECAP parameter is the CL, a pulse width of the neurostimulation is to be modulated. If the ECAP parameter is the peak-to-peak amplitude, a pulse amplitude of the neurostimulation is to be modulated. If the ECAP parameter is the inter-ECAP interval, a pulse frequency of the neurostimulation is to be modulated. Thus, decision tree 1482 maps the input parameters to stimulation parameters to be modulated and types of the modulation.

In various embodiments, a decision tree discussed in this document, such as decision tree 1482, can be established empirically based on simulations with nerve models, data collected from patients, and/or established knowledge relating stimulation parameters to neural response parameters (indicating physiological phenomena). One or more decision trees like decision tree 1482 are predetermined and stored (e.g., in external storage device 818 when system 960 is implemented in external programming device 802) to be used by dynamic pattern composer 962 for determining dynamic stimulation patterns.

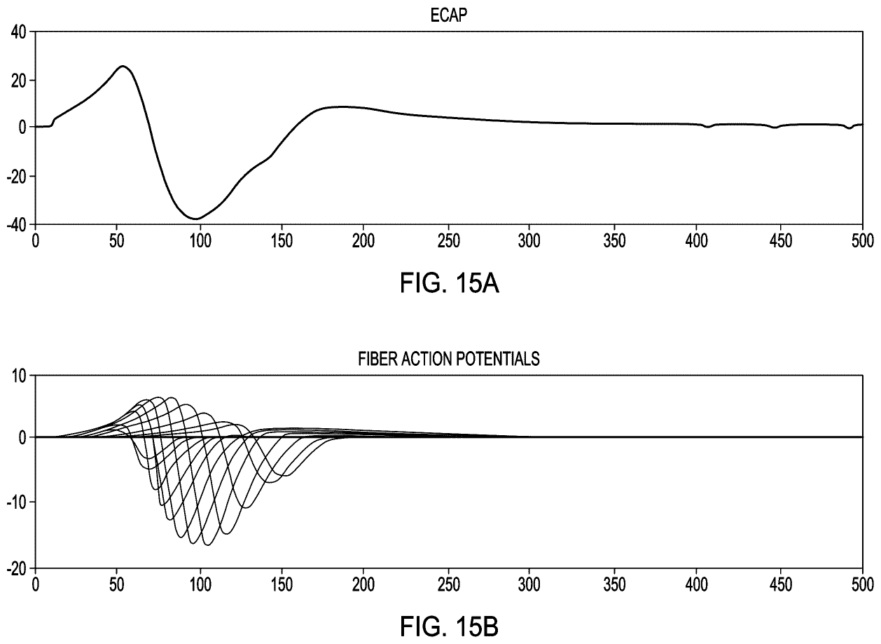

FIG. 15A illustrates an example of an ECAP, and FIG. 15B illustrates an example of the ECAP of FIG. 15A being decomposed to show components of the ECAP attributed to different nerve fiber types. Decomposition of the ECAP can be performed using a neural modeling and computational process (e.g., any methods related to blind source separation or independent component analysis). FIG. 15B shows multiple curves each being an ECAP component attributed to a type of nerve fibers. FIG. 15A shows a single curve being the ECAP that is the sum of the curves shown in FIG. 15B. In various embodiments, it can be of interest to target the neurostimulation on a particular type of nerve fibers to modulate the neural response of this particular type of nerve fibers to the neurostimulation.

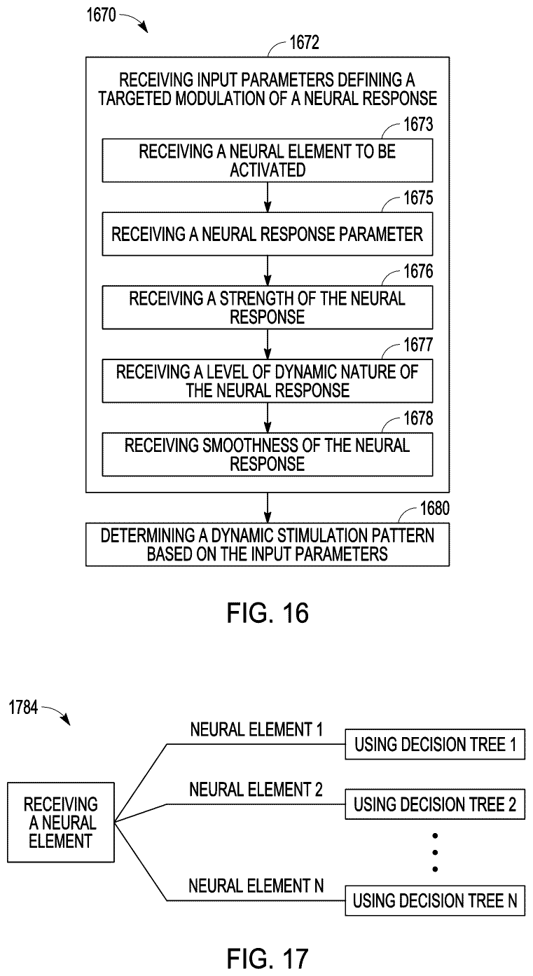

FIG. 16 illustrates an embodiment of a method 1670 for determining a dynamic stimulation pattern for activating a specified neural element, such as may be performed by system 960. Examples of the neural element include various types of nerve fibers (e.g., A-beta, A-delta, and the like). Method 1670 can represent an example of method 1270 when performed for the specified neural element. Dynamic pattern composer 962 can be configured for performing method 1670. In one embodiment, in which system 960 is implemented in external programming device 802, external storage device 818 is configured to be a non-transitory computer-readable storage medium including instructions, which when executed by a system (e.g., system 960 as implemented in external programming device 802), cause the system to perform method 1670.

At 1672, input parameters defining a targeted modulation of a neural response are received. The neural response is a response of the patient to delivery of neurostimulation according to a dynamic stimulation pattern and can be detected from a neural signal sensed from the patient. In various embodiments, the input parameters can define a feature of the neural response (e.g., a neural response parameter) for the specified neural element to be modulated and a degree to which the feature of the neural response is to be modulated. At 1673, a neural element to be activated is received. The neural element is targeted for modulation by the neurostimulation according to the dynamic stimulation pattern and can be specified as a type of nerve fibers (e.g., A-beta, A-delta, and the like). At 1675, a neural response parameter is received. The neural response parameter can be derived from the neural response. At 1676, a strength of the neural response is received. The strength can be represented by, for example, an average value of the neural response parameter. At 1677, a level of dynamic nature of the neural response is received. The level can be represented by a value range of the neural response parameter. In one example, the neural response parameter is an ECAP parameter that can be attributed to the received neural element to be activated (e.g., A-beta fibers). The ECAP parameter can a measure of an ECAP component resulting from activation of A-beta fibers by a neurostimulation pulse (e.g., a peak-to-peak amplitude of the ECAP component) selected from a plurality of ECAP parameters, the strength of the neural response is an average value of the ECAP parameter (e.g., 30 microvolts), and the level of dynamic nature of the neural response is a value range of the ECAP parameter (e.g., 10-50 microvolts). At 1678, smoothness of the neural response is received. The smoothness can be represented by a modulation function applied to a stimulation parameter of the dynamic stimulation pattern. The modulation function can be selected from a plurality of modulation functions (e.g., as supported by dynamic stimulation composer 962) for modulating the stimulation parameter. Examples of the modulation function include random, pseudo-random, sinusoidal, triangular, Poisson distribution, Gaussian distribution, and any other standard or custom functions.

At 1680, the dynamic stimulation pattern is determined based on the input parameters. The dynamic stimulation pattern is defined by stimulation parameters including at least one time-varying stimulation parameter. In various embodiments, the dynamic stimulation pattern is determined based on the input parameters and a decision tree mapping the input parameters to stimulation parameters to be modulated and types of the modulation (e.g., decision tree 1482). In the example in which the neural response parameter is the peak-to-peak amplitude of the ECAP component, the dynamic stimulation patterns determined based on received at 1673, 1675, 1676, 1677, and 1678 includes a neurostimulation pulse sequence with a pulse amplitude to be set and modulated by the sinusoidal modulation function to result in the peak-to-peak amplitude of ECAPs ranging from 10-50 microvolts, with the averaged value of 30 microvolts. The pulse amplitude is selected to be modulated according to a decision tree such as decision tree 1482 or a decision that is further discussed below.

FIG. 17 illustrates an embodiment of a method 1784 for determining a modulation of a stimulation parameter in a dynamic stimulation pattern for a specified neural element. Method 1784 can represent an example of the decision tree used in step 1680 of method 1670. To modulate the neural response parameter of the specified neural element as intended, decision tree 1784 can be used with the input parameters to determine the dynamic stimulation pattern, which is defined by the stimulation parameters including at least one modulated stimulation parameter.

According to method (decision tree) 1784, a neural element to be activated is received. If the neural element is neural element 1 (e.g., A-beta fibers), decision tree 1 (e.g., a decision tree for A-beta fibers) is selected for use in determining determine the dynamic stimulation pattern. If the neural element is neural element 2 (e.g., A-delta fibers), decision tree 2 (e.g., a decision tree for A-delta fibers) is selected for use in determining determine the dynamic stimulation pattern. . . . If the neural element is neural element N, decision tree N is selected for use in determining determine the dynamic stimulation pattern. N is the number of (2 or more) neural elements that can be targeted for activation by the neurostimulation according to the dynamic stimulator pattern. Thus, decision tree 1784 maps the neural elements to be activated to decisions trees for the neural elements, which in turn maps the input parameters except the neural element to stimulation parameters to be modulated and types of the modulation, thereby mapping the input parameters to stimulation parameters to be modulated and types of the modulation.

In various embodiments, decision trees 1-N in method (decision tree) 1784 can be determined for various neural elements 1-N to be activated, depending on the nature of each of the neural elements 1-N and the effect of each stimulation parameter on each neural element. For example, a single decision tree such as decision tree 1482 may be considered sufficient for use as each of decision trees 1-N, or a different decision tree similar to decision tree 1482 may be needed for use as each of decision trees 1-N, depending on the desirable level of control on the modulation of the targeted neural responses for neural elements 1-N.

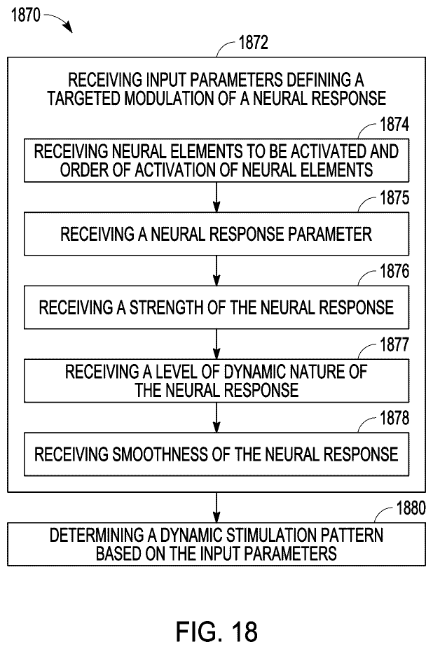

FIG. 18 illustrates an embodiment of a method 1870 for determining a dynamic stimulation pattern for activating multiple specified neural elements in a specified order, such as may be performed by system 960. Examples of the neural elements include various types of nerve fibers (e.g., A-beta, A-delta, and the like). Method 1870 can represent an example of method 1270 when performed for the multiple specified neural elements temporally arranged in the specified order. Dynamic pattern composer 962 can be configured for performing method 1870. In one embodiment, in which system 960 is implemented in external programming device 802, external storage device 818 is configured to be a non-transitory computer-readable storage medium including instructions, which when executed by a system (e.g., system 960 as implemented in external programming device 802), cause the system to perform method 1870.

At 1872, input parameters defining a targeted modulation of a neural response are received. The neural response is a response of the patient to delivery of neurostimulation according to a dynamic stimulation pattern and can be detected from a neural signal sensed from the patient. In various embodiments, the input parameters can define a feature of the neural response (e.g., a neural response parameter) to be modulated for each specified neural element and a degree to which the feature of the neural response is to be modulated. At 1874, multiple neural elements to be activated and an order of activation of these neural elements are received. The neural elements are each targeted for modulation by the neurostimulation according to one or more segments of the dynamic stimulation pattern and can each be specified as a type of nerve fibers (e.g., A-beta, A-delta, and the like). At 1875, a neural response parameter for each neural element is received. The neural response parameter can be derived from the neural response attributed to that neural element. At 1876, a strength of the neural response for each neural element is received. The strength can be represented by, for example, an average value of the neural response parameter. At 1877, a level of dynamic nature of the neural response for each neural element is received. The level can be represented by a value range of the neural response parameter. In one example, the neural response parameter for each neural element is an ECAP parameter that can be attributed to that neural element. The ECAP parameter can be a measure of an ECAP component resulting from activation of each neural element by a neurostimulation pulse (e.g., a peak-to-peak amplitude of the ECAP component) selected from a plurality of ECAP parameters, the strength of the neural response for that neural element is an average value of the ECAP parameter (e.g., 30 microvolts), and the level of dynamic nature of the neural response for that neural element is a value range of the ECAP parameter (e.g., 10-50 microvolts). At 1878, smoothness of the neural response for each neural element is received. The smoothness can be represented by a modulation function applied to a stimulation parameter of the dynamic stimulation pattern. The modulation function can be selected from a plurality of modulation functions (e.g., as supported by dynamic stimulation composer 962) for modulating the stimulation parameter for the one or more segments of the dynamic stimulation pattern for each neural element. Examples of the modulation function include random, pseudo-random, sinusoidal, triangular, Poisson distribution, Gaussian distribution, and any other standard or custom functions.

At 1880, the dynamic stimulation pattern is determined based on the input parameters. The dynamic stimulation pattern is defined by stimulation parameters including at least one time-varying stimulation parameter. In various embodiments, the dynamic stimulation pattern is determined based on the input parameters and a decision tree mapping the input parameters to stimulation parameters to be modulated and types of the modulation for each of the multiple specified neural elements (e.g., decision tree 1784). The dynamic stimulation pattern include windows each associated with one neural element (e.g., fiber type) of the multiple specified neural elements to activate. The windows are temporally arranged according to the order of activation of the multiple specified neural elements.

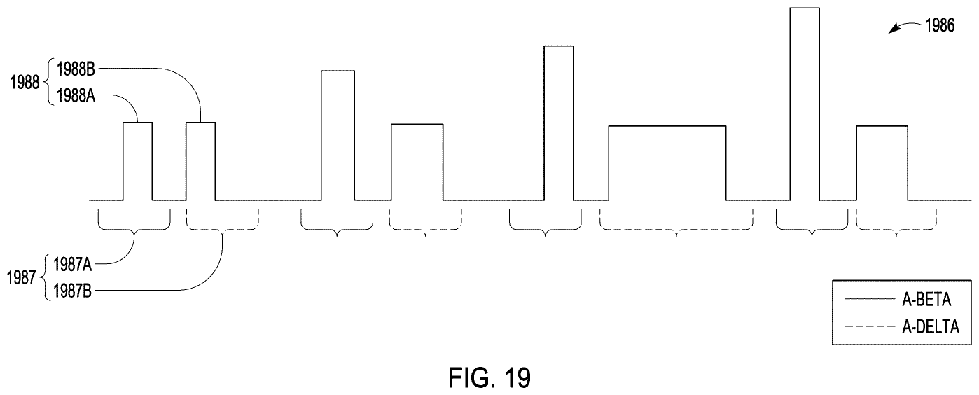

FIG. 19 illustrates an example of a dynamic stimulation pattern 1986 for activating two specified neural elements, such as may be determined using method 1870. The two specified neural elements are A-beta and A-delta fibers, as shown in FIG. 19 for illustrative purposes by way of example and not by way of restriction. Dynamic stimulation pattern 1986 is used for controlling neurostimulation for a sequence of A-fiber activations each followed by an A-delta fiber activation. In the illustrated example, dynamic stimulation pattern 1986 includes windows 1987 and stimulation waveforms 1988 each with a window of windows 1987. Windows 1987 include A-beta windows 1987A and A-delta windows 1987B. Dynamic stimulation pattern 1986 includes a stimulation waveform 1988A for activating A-beta fibers within each of A-beta windows 1987A and a stimulation waveform 1988B for activating A-delta fibers within each of A-delta windows 1987B. The onset time and duration of each window of windows 1987 can be individually programmable. Stimulation waveforms 1988 can be individually programmable.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation to a patient using a stimulation device, the system comprising:
a programming control circuit configured to generate information for programming the stimulation device to control the delivery of the neurostimulation according to a dynamic stimulation pattern defined by stimulation parameters including at least one time-varying stimulation parameter; and
a stimulation programming circuit including a dynamic pattern composer configured to determine the dynamic stimulation pattern based on a targeted modulation of a neural response by the neurostimulation according to the dynamic stimulation pattern, the neural response being a response of the patient to the delivery of the neurostimulation.

2. The system of claim 1, wherein the programming control circuit is further configured to generate the information for programming the stimulation device to control the delivery of the neurostimulation according to a stimulation program including multiple stimulation patterns and a schedule specifying an onset time and a duration of each stimulation pattern of the multiple stimulation patterns, the multiple stimulation patterns defined by the stimulation parameters and including the dynamic stimulation pattern, and the stimulation programming circuit is configured to determine the stimulation program.

3. The system of claim 2, comprising a stimulation device and a programming device configured to program the stimulation device and including the programming control circuit and the stimulation programming circuit, the stimulation device configured to sense a neural signal including the neural response, and wherein the dynamic pattern composer is configured to allow for determination of one or more features of the neural response to be modulated by the neurostimulation according to the dynamic stimulation pattern.

4. The system of claim 1, wherein the dynamic pattern composer is configured to receive input parameters defining the targeted modulation of the neural response by the neurostimulation according to the dynamic stimulation pattern and to determine the dynamic stimulation pattern based on the input parameters.

5. The system of claim 4, wherein the dynamic pattern composer is configured to receive a neural response parameter of the input parameters by selecting the neural response parameter from a plurality of neural response parameters to be modulated by the neurostimulation according to the dynamic stimulation pattern, the neural response parameters each being a measure of the neural response and derivable from a sensed neural signal including the neural response.

6. The system of claim 5, wherein the dynamic pattern composer is further configured to receive one or more quantitative parameters of the input parameters, the one or more quantitative parameters defining a degree to which the neural response parameter is to be modulated by the neurostimulation.

7. The system of claim 6, wherein the dynamic pattern composer is configured to receive the neural response parameter being an evoked compound action potential (ECAP) parameter and the one or more quantitative parameters including a strength of the neural response, a level of dynamic nature of the neural response, and smoothness of the neural response, the ECAP parameter measurable from one or more ECAP features each being a morphological feature of ECAPs in the sensed neural signal, the smoothness defined by a modulation function to be applied to produce the time-varying stimulation parameter by modulating a stimulation parameter of the stimulation parameters defining the dynamic stimulation pattern.

8. The system of claim 5, wherein the dynamic pattern composer is configured to determine the dynamic stimulation pattern with at least one stimulation parameter of the stimulation parameters being modulated using the input parameters and a decision tree mapping plurality of neural response parameters to stimulation parameters to be modulated and types of modulation.

9. The system of claim 8, wherein the dynamic pattern composer is further configured to receive a neural element to activate as an additional input parameter of the input parameters and to determine the dynamic stimulation pattern for activating the neural element by delivering the neurostimulation according to the dynamic stimulation pattern using the input parameters and a decision tree mapping plurality of neural response parameters to stimulation parameters to be modulated and types of modulation for the neural element.

10. The system of claim 8, wherein the dynamic pattern composer is further configured to receive multiple neural elements to activate and an order of activation of the multiple neural elements as additional input parameters of the input parameters and to determine the dynamic stimulation pattern for activating each neural element of the multiple neural elements by delivering the neurostimulation according to the dynamic stimulation pattern using the input parameters and a decision tree mapping plurality of neural response parameters to stimulation parameters to be modulated and types of modulation for the each neural element, the dynamic stimulation pattern including windows each associated with one neural element of the multiple neural elements to activate and temporally arranged according to the order of activation of the multiple neural elements.

11. A method for delivering neurostimulation to a patient using a stimulation device, the method comprising:
determining, using a processor of a programming device, a dynamic stimulation pattern based on a targeted modulation of a neural response by the neurostimulation according to the dynamic stimulation pattern, the dynamic stimulation pattern defined by stimulation parameters including at least one time-varying stimulation parameter, the neural response being a response of the patient to the delivery of the neurostimulation; and
programming a stimulation device, using the programming device, to deliver the neurostimulation and control the delivery of the neurostimulation according to the determined dynamic stimulation pattern.

12. The method of claim 11, wherein determining the dynamic stimulation pattern comprises:
receiving input parameters defining the targeted modulation of the neural response by the neurostimulation; and
determining the dynamic stimulation pattern based on the input parameters.

13. The method of claim 12, wherein receiving the input parameters comprises receiving a neural response parameter selected from a plurality of neural response parameters to be modulated by the neurostimulation, the neural response parameters each being a measure of the neural response and derivable from a sensed neural signal including the neural response.

14. The method of claim 13, wherein receiving the input parameters further comprises receiving one or more quantitative parameters defining a degree to which the neural response parameter is to be modulated by the neurostimulation.

15. The method of claim 14, wherein receiving the neural response parameter comprises receiving an evoked compound action potential (ECAP) parameter, the ECAP parameter measurable from one or more ECAP features each being a morphological feature of ECAPs in the sensed neural signal.

16. The method of claim 14, wherein receiving the one or more quantitative parameters comprises receiving a modulation function to be applied to produce the time-varying stimulation parameter by modulating a stimulation parameter of the stimulation parameters defining the dynamic stimulation pattern.

17. The method of claim 12, wherein determining the dynamic stimulation pattern comprises determining the dynamic stimulation pattern with at least one stimulation parameter of the stimulation parameters being modulated using the input parameters and a decision tree mapping the plurality of neural response parameters to stimulation parameters to be modulated and types of modulation.

18. The method of claim 17, wherein receiving the input parameters further comprises receiving a neural element to activate, and determining the dynamic stimulation pattern comprises determining the dynamic stimulation pattern for activating the neural element by delivering the neurostimulation according to the dynamic stimulation pattern using the input parameters and a decision tree mapping plurality of neural response parameters to stimulation parameters to be modulated and types of modulation for the neural element.

19. The method of claim 17, wherein receiving the input parameters further comprises receiving multiple neural elements to activate and an order of activation of the multiple neural elements, and determining the dynamic stimulation pattern comprises determining the dynamic stimulation pattern for activating each neural element of the multiple neural elements by delivering the neurostimulation according to the dynamic stimulation pattern using the input parameters and a decision tree mapping plurality of neural response parameters to stimulation parameters to be modulated and types of modulation for the each neural element, the dynamic stimulation pattern including windows each associated with one neural element of the multiple neural elements to activate and temporally arranged according to the order of activation of the neural elements.

20. A non-transitory computer-readable storage medium including instructions, which when executed by a system, cause the system to perform a method for delivering neurostimulation from a stimulation device to a patient, the method comprising:
determining a dynamic stimulation pattern based on a targeted modulation of a neural response by the neurostimulation according to the dynamic stimulation pattern, the dynamic stimulation pattern defined by stimulation parameters including at least one time-varying stimulation parameter, the neural response being a response of the patient to the delivery of the neurostimulation; and
programming the stimulation device to deliver the neurostimulation and to control the delivery of the neurostimulation according to the determined dynamic stimulation pattern.
